(19) United States
(12) Patent Application Publication
(10) Pub. No.: US 2007/0297996 A1
(43) Pub. Date: Dec. 27, 2007

(54) Multi-phase composition comprising a sunscreen

(75) Inventor: Paul Robert Tanner, Lebanon, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company

(21) Appl. No.: 11/472,737

(22) Filed: Jun. 22, 2006

(51) Int. Cl. A61K 8/89; A61K 8/49; A61K 8/37

(52) U.S. Cl. 424/59

(57) ABSTRACT

Personal care composition comprising a discrete transparent aqueous phase and a discrete non-aqueous phase, wherein the aqueous phase comprises from about 0.05% to about 5% of a polymeric thickener, and from about 50% to about 99.5% water, both by weight of the aqueous phase; and the non-aqueous phase comprises from about 20% to about 90% of a sunscreen selected from the group consisting of oil-soluble sunscreens, oil-insoluble sunscreens, and combinations thereof; and from about 5% to about 40% of an oil thickener, all by weight of the non-aqueous phase; and wherein the ratio of the weight of the aqueous phase to the weight of the non-aqueous phase is from about 1:1 to about 9:1; and wherein the aqueous phase and the non-aqueous phase are visually distinct and form a stable pattern.

### FIELD OF THE INVENTION

The present invention relates to a stable composition comprising at least two visually distinct phases, wherein at least one phase comprises an oil-soluble sunscreen.

### BACKGROUND OF THE INVENTION

An ongoing need exists to provide personal care compositions which prevent damage to the skin and other keratinous tissue from harmful ultraviolet radiation. To provide effective protection, such compositions must be regularly applied. An additional ongoing need exists, therefore, to provide sunscreen compositions that have a pleasant appearance and feel, and thus encourage frequent use. Multi-phase compositions, for example products in which distinct stripes or swirled patterns are visible, have an especially desirable appearance. One example of such a composition comprises at least one patterned non-aqueous phase, for example an internal opaque and/or colored spiral or helix shape, surrounded by a transparent, external aqueous phase. Because patterned, multi-phase compositions tend to be well-accepted by consumers, and may be more frequently used, it would be desirable to provide such a composition comprising a sunscreen active. Having one or more sunscreen actives in both phases of the composition may provide a more even distribution of the active and thus maximum protective benefit. However, formulation of patterned, multi-phase sunscreen compositions has proven difficult for a number of reasons.

To remain stable and visibly distinct, both phases of the composition must be sufficiently thickened. Thickening aqueous compositions to produce compositions acceptable to consumers is relatively straightforward; however, thickening oil-based compositions remains challenging. Oil-soluble sunscreen compounds, desirable for their effectiveness, tend to be particularly difficult to thicken while maintaining solubility and a pleasant feel. Whereas it may be possible to use various types of waxy materials to thicken an oil phase, these may exhibit instability at higher temperatures. Oil-soluble sunscreen compounds also may exhibit limited solubility (i.e. crystallize) when used in an effective amount with traditional thickeners.

In addition to sufficient thickening, it also is necessary to minimize the chemical interaction between the aqueous and non-aqueous phases. For example, migration of water between the phases results in a loss of phase integrity, and thus of the appearance of the pattern. This may decrease the visual appeal of the pattern.

There exists a need, therefore, to provide a multi-phase, patterned sunscreen composition in which the chemical interaction of the phases is mimimized to provide stability, and which has a pleasant feel and appearance so as to encourage frequent use.

### SUMMARY OF THE INVENTION

The present invention meets the aforementioned needs by providing a patterned, non-emulsified composition having at least one non-aqueous phase and an aqueous phase. The non-aqueous phase comprises an oil-soluble sunscreen and oil thickeners which are stable at higher temperatures. The composition of the present invention provides a number of advantages. Because the composition is non-emulsified, there is no need for emulsifiers which tend to have a poor skin feel. The oil thickeners provide a pleasant skin feel, and minimize the migration of water into the oil phase, thus stabilizing the visually appealing pattern. When the composition does not comprise waxes as primary thickeners, the composition is stable at relatively high temperatures, and avoids the need for heating during the manufacturing process (i.e. a “cold-filling” process may be used), thus providing a lower cost and increased efficiency of production. In addition, the visual appeal encourages initial use while the pleasant feel provides an immediate benefit. This in turn encourages frequent use of a sunscreen active, which provides a chronic benefit in the form of protection from harmful UV-radiation.

The following describe some non-limiting embodiments of the present invention.

According to a first embodiment, a stable personal care composition is described comprising a discrete transparent aqueous phase and a discrete non-aqueous phase. The aqueous phase comprises from about 0.05% to about 5% of a polymeric thickener, and from about 50% to about 99.5% water. The non-aqueous phase comprises from about 20% to about 90% of a sunscreen selected from the group consisting of oil-soluble sunscreens, oil-insoluble sunscreens, and combinations thereof; and from about 5% to about 40% of an oil thickener. The ratio of the weight of the aqueous phase to the weight of the non-aqueous phase is from about 1:1 to about 9:1, and the aqueous phase and the non-aqueous phase are visually distinct and form a stable pattern.

According to yet another embodiment of the present invention, a method of providing a benefit to mammalian keratinous tissue in need thereof is provided, comprising the step of topically applying to mammalian keratinous tissue the composition of the first embodiment.

According to yet another embodiment of the present invention, a kit is described comprising a composition according to the first embodiment.

### DETAILED DESCRIPTION OF THE INVENTION

The present invention describes a stable, non-emulsified multi-phase personal care composition comprising a sunscreen. The composition may be used in skin care, cosmetics, and hair care products, non-limiting uses of which include moisturizers, conditioners, anti-aging compounds, skin lightening compounds, and combinations thereof. In one embodiment, the composition is applied to the face, neck and other exposed areas of the body. Alternatively, the composition is applied to insult-affected areas of keratinous tissue.

In all embodiments of the present invention, all percentages are by weight of the phase, i.e. the phase in which the percentage of the relevant compound is found, unless otherwise specified. All ratios are weight ratios, unless specifically stated otherwise. The number of significant digits conveys neither limitations on the indicated amounts nor on the accuracy of the measurements. All amounts indicating quantities, percentages, proportions, etc. are understood to be modified by the word “about” unless otherwise specifically indicated. All measurements are understood to be made at about 25° C. and at ambient conditions, where “ambient conditions” means conditions under about one atmosphere of pressure and at about 50% relative humidity.

Herein, “personal care composition” means compositions suitable for topical application on mammalian keratinous tissue. The personal care compositions described herein may contain one or more skin care actives. “Skin care actives,” or “actives,” as used herein, means compounds that aid in regulating the condition of skin and of other mammalian keratinous tissue, for example, by providing a benefit or improvement to the keratinous tissue.

“Keratinous tissue,” as used herein, refers to keratin-containing layers disposed as the outermost protective covering of mammals which includes, but is not limited to, skin, hair, nails, cuticles, etc.

Herein, “stable” and “stability” mean compositions which are substantially unaltered in chemical state, physical homogeneity and/or color, and which comprise at least two discrete, visually distinct phases upon exposure to conditions reasonably expected to be incurred in shipping, storage and use, for example, for at least 30 days at a temperature of from about 0° C. to about 40° C.

“Visually distinct,” as used herein, means that the phases are visually distinguishable as two separate phases without the aid of magnification by one having substantially unimpaired vision.

“Discrete phases,” as used herein, means that the phases do not exhibit blurring, bleeding and/or intermingling with respect to each other, as would be discernible without the aid of magnification by one having substantially unimpaired vision, when the phases are in substantially continuous contact.

“Phase” as used herein means a homogeneous portion of the composition, which is physically distinguishable from one or more other phases by a difference in appearance (color, opacity), viscosity, hydrophobicity, ingredients, etc. as determined by known methods to one of skill in the art. In one embodiment, the phases are visually distinguishable when viewed through a transparent packaging.

“Derivatives,” as used herein, means ester, ether, amide and/or salt derivatives of the relevant compound.

“Substantially free,” as used herein, unless otherwise specified means that the compound of which the composition or phase is substantially free is not added to the composition or phase; however, small amounts incidentally may be present, for example as a by-product of chemical reactions, or as a result of absorption from the surroundings. Alternatively, substantially free may be understood to mean less than about 1%, by weight of the phase.

Herein, “regulating the condition of keratinous tissue” means improving the condition of mammalian keratinous tissue and/or prophylactically regulating the condition of mammalian keratinous tissue, and includes, for example, protecting the tissue from ultraviolet radiation, and regulating the signs of skin aging. Herein, “improving the condition of mammalian keratinous tissue” means effecting a visually and/or tactilely perceptible positive change in the appearance and feel of the tissue. Conditions that may be regulated and/or improved include, but are not limited to, one or more of the following: increasing the luminosity, or “glow” of the skin, reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, dryness, brittleness, and graying hair.

As used herein, “signs of skin aging,” include, but are not limited to, outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; flaking; dryness; loss of skin elasticity; discoloration (including undereye circles); blotchiness; dullness or lack of luminosity, sallowness; hyperpigmented skin regions such as age spots and freckles; keratoses; abnormal differentiation; hyperkeratinization; elastosis; collagen breakdown, and other histological changes in the stratum comeum, dermis, epidermis, vascular system (e.g., telangiectasia or spider vessels), and underlying tissues (e.g., fat and/or muscle), especially those proximate to the skin.

“Dermatologically-acceptable,” as used herein, means that the compositions or components thereof so described are suitable for use in contact with mammalian keratinous tissue without undue toxicity, incompatibility, instability, allergic response, and the like.

“Insult-affected keratinous tissue,” as used herein, means keratinous tissue which exhibits discomfort, irritation, an unpleasant or irregular appearance and the like, for example after exposure to a physical and/or chemical irritant. Non-limiting examples of insult-affected keratinous tissue include sunburn and other types of burns; rashes, such as diaper rash, shaving rash and allergen-induced rashes; discoloration, such as bleaching, staining or hyperpigmentation; nicks and cuts due to, for example, shaving; dry, chapped or rough skin due to exposure to example wind, cold and/or low humidity, etc. Non-limiting examples of insults include ultraviolet radiation, wind, low humidity, allergens, pollutants, chemical and natural irritants, bodily fluids, bodily waste, excessive moisture, bacteria, fungi, etc.

Herein “kit” means a packaging unit comprising at least one composition described herein. The kit may comprise an outer packaging unit, which in turn may comprise one or more inner packaging units. The inner and outer packaging units may be of any type suitable for containing, presenting and/or reasonably protecting from damage the contents of the kit. The kit may comprise a plurality of components, such as a multi-phase composition as described herein. The kit further may comprise one or more additional compositions, suitable for application prior to or after application of the composition described herein, one or more orally ingestible dietary supplements, a delivery enhancement device, instructions for use of the device, instructions for complying with suitable application regimens, and combinations thereof.

### I. Composition

The composition of the present invention comprises at least one discrete, visually distinct first phase and at least one discrete, visually distinct second phase. In one embodiment, the first phase is a transparent, clear or translucent aqueous phase and the second phase is either an opaque white or a colored non-aqueous phase. In one embodiment, at least one non-aqueous phase forms a stable pattern, for example a continuous or discontinous line, a spiral, a curve, or other geometric shape, within a transparent aqueous phase, where “within” means that the non-aqueous phase is substantially surrounded by the aqueous phase and does not contact the side of a container. Alternatively, a plurality of non-aqueous phases forms a pattern within a transparent aqueous phase, for example, a multiple helix. The non-aqueous phases may be similar in appearance, or may differ in appearance and/or in composition, provided they fall within the parameters for the non-aqueous phase set forth herein. Alternatively, the aqueous and non-aqueous phase(s) together may form a swirled pattern, wherein both phases alternately contact the side of a container and wherein the width of each of the phases, when viewed through the side of a transparent container, is substantially constant, though may differ from each other. Alternatively, the aqueous and non-aqueous phase(s) together may form a marbled pattern, wherein the phases alternately contact the side of the container and wherein the width of the individual phases, when viewed through the side of a transparent container, may vary throughout the composition. In another alternative embodiment, at least one aqueous phase forms a pattern within a non-aqueous phase.

The composition optionally may comprise a third visually distinct and stable phase. The third phase may be a substantially anhydrous phase comprising silicone elastomers, and alternatively may be an emulsion. In one embodiment, the third phase is patterned and within the transparent aqueous phase. Alternatively, the third phase may form part of a swirled and/or marbled pattern.

The viscosity of the aqueous, non-aqueous phase and optional third phase is greater than about 30,000 and alternatively greater than about 50,000 centipoise (cps). In one embodiment, the viscosity is from about 30,000 cps to about 1 million cps, and alternatively from about 50,000 cps to about 500,000 cps.

The ratio of the weight of the aqueous phase to the total weight of one or more non-aqueous phases is about 1:1, alternatively about 4:1, and alternatively about 9:1. In one embodiment, the composition comprises an aqueous phase and one or more non-aqueous phases wherein the ratio of the weight of the aqueous phase to the total weight of the non-aqueous phase(s) is about 4:1. In one embodiment, the composition comprises an aqueous phase, one or more non-aqueous phases, and one or more third phases, wherein the ratio of the weight of the aqueous phase to the total weight of the non-aqueous phase(s) and third phase(s) is about 1:1, alternatively about 4:1 and alternatively about 9:1.

When one or more phases forms a shape such as a line, spiral, helix, multiple helix, curve, etc. the phase may have a substantially constant average width and/or diameter, from about 0.5 mm to about 30 mm, and alternatively from about 1 mm to about 10 mm.

In one embodiment, the width of the non-aqueous phase and the ratio of the weight of the aqueous phase to the weight of the non-aqueous phase, are such that the composition is evenly distributed upon dispensing from the packaging. “Evenly distributed” is understood to mean that the dispensed composition has a ratio of the weight of the aqueous phase to the total weight of the non-aqueous phase(s) that is substantially the same as the ratio in the packaging, prior to dispensing. Additionally or alternatively, the width of the non-aqueous phase and the ratio of the weight of the aqueous phase to the weight of the non-aqueous phase, are such that an effective amount of a sunscreen and/or additional skin care active is present in the composition upon a single dispensation from the container.

In one embodiment, the composition is substantially free from surfactants, and/or emulsifiers. Alternatively, the non-aqueous phase is substantially free from surfactants and/or emulsifiers.

In one embodiment, the composition is substantially free from waxes. When the composition is substantially free from waxes, and alternatively comprises less than about 1% by weight of the phase of one or more waxes, the composition may exhibit stability at a temperature of up to about 45° C., and the manufacturing process need not comprise the step of heating the composition above room temperature, or about 25° C. (also referred to as “cold processing.”) The patterned compositions may be made by a process in which the phases are added to the container via one or more nozzles, and in which the nozzle(s) and the container may be moved with respect to each other during the filling process. Suitable processes are described in U.S. Pat. Nos. 6,213,166; 6,245,344; 6,367,519; 6,516,838; and in U.S. patent publication 2003/0111130.

### A. Non-Aqueous (Oil) Phase

The composition of the present invention comprises at least one non-aqueous, or oil, phase. The non-aqueous phase comprises at least one oil-soluble sunscreen, a polar solvent, and an oil thickener. The non-aqueous phase further may comprise at least one oil-soluble skin care active, an oil-soluble colorant, one or more insoluble components, such as a sunscreen, colorant, powder, particulate material, or additional skin care active, and may be substantially anhydrous. Alternatively the oil phase may comprise less than about 5%, and alternatively less than about 1%, of water.

### 1. Organic Sunscreen

The non-aqueous phase of the composition of the present invention may comprise from about 20% to about 90%, alternatively from about 30% to about 80%, and alternatively from about 40% to about 70%, by weight of the non-aqueous phase, of an oil-soluble sunscreen, an oil-insoluble sunscreen, and mixtures thereof. Herein, “sunscreen” is understood to include both sunscreens and UV-light absorbers.

Non-limiting examples of suitable oil-soluble sunscreens are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93 and include benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof.

Non-limiting examples of suitable oil-insoluble sunscreens include methylene bis-benzotriazolyl tetramethylbutyl-phenol, titanium dioxide, zinc cerium oxide, zinc oxide, and derivatives and mixtures thereof. It is to be understood that “oil-insoluble sunscreen” does not encompass water-soluble sunscreens.

### 2. Polar Solvent

The non-aqueous phase of the composition of the present invention may comprise from about 0.001% to about 30%, and alternatively from about 5% to about 25%, of a polar solvent. “Polar,” as used herein, means that the solvent has an average solubility parameter of from about 7 to about 13 (cal/cm³)^{0.5}, and alternatively from about 8 to about 12 (cal/cm³)^{0.5}, where “cal” means calories. Oils having a lower solubility parameter than 7 and higher than 13 may be used if, when the oils are blended with other oils, the weighted average of the solubility parameter of the oil blend is from about 7 to about 13. Solubility parameters are discussed in more detail by C. D. Vaughan in “The Solubility Parameter: What is it?,” Cosmetics & Toiletries vol. 106, November, 1991, pp. 69-72.

Non-limiting examples of suitable polar solvents include esters of mono and dibasic carboxylic acids with mono and polyhydric alcohols; polyoxyethylenes, polyoxypropylenes, mixtures of polyoxyethylene and polyoxypropylene ethers of fatty alcohols; and mixtures thereof. Alternatively, the polar solvent is selected from the group consisting of esters and ethers. Alternatively, the polar solvent is selected from the group consisting of di-isopropyl adipate, butyl and isopropyl phthalimide (Pelemol™ BIP), phenylethyl benzoate (X-tend™ 226), dicaprylyl carbonate (Tegosoft™ DEC), isopropyl lauroylsarcosinate (Eldewr™ SL 205), isononyl isononanoate, butyl octylsalicylate (Hallbrite™ BHB), dioctyl malate, dicaprylyl maleate (Hallbrite™ DCM), isopropyl isostearate, propylene glycol dicaprate, esters of C12-C15 alcohol benzoates (e.g. Finsolv™ TN supplied by Finetex™), and derivatives and mixtures thereof.

### 3. Oil Thickener

The non-aqueous phase of the composition of the present invention may comprise from about 5% to about 40%, and alternatively from about 7% to about 30%, and alternatively from about 8% to about 25%, by weight of the non-aqueous phase, of an oil thickener. Non-limiting examples of suitable particulate oil thickeners include silica, hydrophobically-modified silica, fumed silica, hydrophobically-modified fiumed silica; polyethylene, polymethylstyrene, polypropylene, polystyrene, polyurethane, nylon, polytetrafluoroethylene (PTFE); boron nitride; and derivatives and mixtures thereof. In one embodiment, the oil thickener is selected from the group consisting of silica, hydrophobically-modified silica, fumed silica, hydrophobically-modified fumed silica, polytetrafluoroethylene, polyethylene, and derivatives and mixtures thereof.

Fumed silica is produced in a hydrogen gas flame according to the reaction SiCl₄+2H₂+O₂→SiO₂+4HCl. Untreated fumed silica contains hydrophilic surface silanol and siloxane groups. Hydrophobic fumed silica is formed by chemical reaction of the surface silanol and siloxane groups with hydrophobic modifying compounds. Suitable hydrophobic modifying compounds include, but are not limited to, dimethyl silyl, trimethylsiloxyl and dimethicone, to produce respectively silica dimethyl silylate, silica silylate, and silica dimethicone silylate. Fumed silicas are described by S. Hasenzahl and A. Braunagel in “Fumed silica for personal care and cosmetics—versatile and effective,” SOFW-Journal, vol. 129, issue 8 (2003), p. 2-8.

Suitable hydrophobic modified silicas include, but are not limited to, the Aerosil R series (wherein “R” means “repellent”), for example, Aerosil R805, R812, R972, R974, R976, R976 S, R8200, RX200 and RX300, available from Degussa AG; Cab-o-sil TS-530, TS-610, TS-720, available from Cabot Corp.; and HDK H2000, H15, H18, H20 and H30, available from Wacker-chemie GmbH, and mixtures thereof. In one embodiment, the hydrophobic modified silica of the present invention is selected from the group consisting of silica dimethyl silylate, silica silylate, silica dimethicone silylate, and combinations thereof. Alternatively, the hydrophobic modified silica is selected from the group consisting of silica silylate, silica dimethicone silylate and combinations thereof. In one embodiment, the hydrophobic modified silica has a carbon content of about 1.5% or greater, by weight of the hydrophobically-treated fumed silica. Alternatively, the hydrophobic modified silica has a carbon content of about 2.0% or greater, by weight of the hydrophobically-treated fumed silica.

### B. Aqueous Phase

The composition of the present invention comprises at least one aqueous, or water, phase. The aqueous phase comprises a polymeric thickening agent, and may comprise a water-soluble sunscreen, from about 50% to about 99.5% by weight of the aqueous phase of water, from about 0.001% to about 25% by weight of the aqueous phase of a conditioning agent, such as glycerin, a water-soluble skin care active, and other optional ingredients including a pH adjuster, water-soluble colorants, etc.

### 1. Polymeric Thickening Agent

The composition of the present invention may comprise from about 0.05% to about 5%, alternatively from about 0.2% to about 4%, and alternatively from about 0.25% to about 3%, by weight of the aqueous phase, of one or more polymeric thickening agents, including natural and synthetic polymeric thickening agents and gelling agents. “Polymeric thickening agents” is understood herein to include both polymers and copolymers. Nonlimiting classes of thickening agents include crosslinked polyacrylate polymers and copolymers, polyacrylamide polymers and copolymers, polysaccharides, gums, and salts, derivatives and mixtures thereof.

### 2. Water-Soluble Sunscreen

The aqueous phase of the composition of the present invention may comprise from about 0.001% to about 5%, and alternatively from about 0.2% to about 4%, of a water-soluble, organic sunscreen. Non-limiting examples of suitable sunscreens include phenylbenzimidazole sulfonic acid (PBSA), terephthalylidene dicamphor sulfonic acid, (Mexoryl™ SX), benzophenone-4, benzophenone-5, benzylidene camphor sulfonic acid, cinnamidopropyl-trimonium chloride, methoxycinnamido-propyl ethyldimonium chloride ether, disodium bisethylphenyl triaminotriazine stilbenedisulfonate, disodium distyrylbiphenyl disulfonate, disodium phenyl dibenzimidazole tetrasulfonate, methoxycinnamido-propyl hydroxysultaine, methoxycinnamido-propyl laurdimonium tosylate, PEG-25 PABA (p-aminobenzoic acid), polyquaternium-59, TEA-salicylate, and salts, derivatives and mixtures thereof.

### C. Optional Third Phase

The composition of the present invention may comprise third phase. The third phase may be substantially anhydrous, and comprise silicone elastomers, useful for reducing the tackiness of the composition and for providing a pleasant feel upon application. Alternatively, the third phase may be an emulsion, and alternatively, an oil-in-water emulsion. The third phase further may comprise at least one additional skin care active, a colorant, and mixtures thereof.

Non-limiting examples of useful silicone elastomers are described in U.S. patent publication 2003/0049212A1, and include crosslinked organopolysiloxane (or siloxane) elastomers. Non-limiting examples of suitable crosslinked organopolysiloxane elastomers include dimethicone crosspolymers, dimethicone/vinyl dimethicone crosspolymers, PEG-12 dimethicone crosspolymers, and copolymers, derivatives and combinations thereof, supplied by Dow Corning™, C30-45 alkyl cetearyl dimethicone crosspolymer, cetearyl dimethicone crosspolymer, and copolymers, derivatives and mixtures thereof, supplied by General Electric™; dimethicone/phenyl vinyl dimethicone crosspolymer, dimethicone/PEG-10/15crosspolymer, dimethicone/PEG-10 crosspolymer, PEG-15/lauryl dimethicone crosspolymer, vinyl dimethicone/lauryl dimethicone crosspolymer, trifluoropropyl dimethicone/trifluoropropyl divinyldimethicone crosspolymer, trifluoropropyl dimethicone/PEG-10 crosspolymer, dimethicone/polyglycerin-3 crosspolymer, lauryl dimethicone/polyglycerin-3 crosspolymer, and copolymers, derivatives and mixtures thereof, supplied by Shin EtSu™; and mixtures of any of the above.

The elastomer phase may comprise a suitable solvent for the crosslinked organopolysiloxane elastomers. Non-limiting examples of suitable solvents are described in U.S. patent publication 2003/0049212A1. The concentration of the solvent in the cosmetic compositions of the present invention may vary depending upon the type and amount of solvent and the cross-linked siloxane elastomer employed, and when combined with the cross-linked organopolysiloxane elastomer particles of the present invention, suspends and swells the elastomer particles to provide an elastic, gel-like network or matrix.

### D. Optional Components

### 1. Colorants

The aqueous, non-aqueous and/or optional third phase of the composition of the present invention may comprise from about 0.001% to about 25%, alternatively from about 0.001% to about 25%, and alternatively from about 0.01% to about 5%, of a suitable colorant. Non-limiting classes of suitable colorants include, but are not limited to organic and/or inorganic pigments, natural and/or synthetic dyes, lakes, including FD&C and/or D&C lakes and blends, and mixtures of any of the foregoing. When the colorant is in the non-aqueous phase or elastomer phase, the colorant may be oil-soluble, or alternatively surface-coated with a hydrophobic coating. When the colorant is in the aqueous phase, the colorant may be water soluble or alternatively surface coated with a hydrophilic coating.

Non-limiting examples of suitable colorants include iron oxides, ferric ammonium ferrocyanide, manganese violet, ultramarine blue, and chromium oxide, phthalocyanine blue and green pigment, encapsulated dyes, inorganic white pigments, for example TiO₂, ZnO, or ZrO₂, FD&C dyes, D&C dyes, and mixtures thereof.

Also useful herein are interference pigments, including hydrophobically-modified interference pigments. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃ and include pearlescent pigments. Intereference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

### 2. Additional Skin Care Actives

The aqueous, non-aqueous and/or third elastomer phase of the composition of the present invention may comprise at least one skin care active. Solubility in water and oil is within the knowledge of one of skill in the art, and can be determined using known methods of analysis. One of skill in the art further will understand that solubility may be affected by the type and concentration of other components in the composition, and by conditions such as pH and ionic strength. Many skin care actives may provide more than one benefit, or operate via more than one mode of action; therefore, classifications herein are made for the sake of convenience and are not intended to limit the active to that particular application or applications listed. All percentages are based on the weight of the phase, aqueous or non-aqueous, into which the active partitions.

### Vitamins

The compositions of the present invention may comprise from about 0.0001% to about 50%, alternatively from about 0.001% to about 10%, alternatively from about 0.01% to about 5%, of at least one vitamin. Herein, “vitamins” means vitamins, pro-vitamins, and their salts, isomers and derivatives. Non-limiting examples of suitable vitamins include: vitamin B compounds (including B1 compounds, B2 compounds, B3 compounds such as niacinamide, niacinnicotinic acid, tocopheryl nicotinate, C1-C18 nicotinic acid esters, and nicotinyl alcohol; B5 compounds, such as panthenol or “pro-B5”, pantothenic acid, pantothenyl; B6 compounds, such as pyroxidine, pyridoxal, pyridoxamine; carnitine, thiamine, riboflavin); vitamin A compounds, and all natural and/or synthetic analogs of Vitamin A, including retinoids, retinol, retinyl acetate, retinyl palmitate, retinoic acid, retinaldehyde, retinyl propionate, carotenoids (pro-vitamin A), and other compounds which possess the biological activity of Vitamin A; vitamin D compounds; vitamin K compounds; vitamin E compounds, or tocopherol, including tocopherol sorbate, tocopherol acetate, other esters of tocopherol and tocopheryl compounds; vitamin C compounds, including ascorbate, ascorbyl esters of fatty acids, and ascorbic acid derivatives, for example, ascorbyl phosphates such as magnesium ascorbyl phosphate and sodium ascorbyl phosphate, ascorbyl glucoside, and ascorbyl sorbate; and vitamin F compounds, such as saturated and/or unsaturated fatty acids. In one embodiment, the composition comprises a vitamin selected from the group consisting of vitamin B compounds, vitamin C compounds, vitamin E compounds and mixtures thereof. Alternatively, the vitamin is selected from the group consisting of niacinamide, tocopheryl nicotinate, pyroxidine, panthenol, vitamin E, vitamin E acetate, ascorbyl phosphates, ascorbyl glucoside, and mixtures thereof.

### Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, carnosine (beta-alanine-histidine), palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, acetyl-glutamate-glutamate-methionine-glutamine-arginine-arginine (Ac-EEMQRR; Argireline®), and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

### Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-glucosamine, and also those described in PCT Publication WO 02/076423 and U.S. Pat. No. 6,159,485, issued to Yu, et al. In one embodiment, the composition comprises from about 0.01% to about 15%, alternatively from about0.1% to about 10%, and alternatively from about 0.5% to about 5%, of the sugar amine.

### Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide or tocopheryl nicotinate), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

### Particulates

The compositions of the present invention may comprise one or more particulate materials, non-limiting examples of which include inorganic powders (for example, iron oxides, zinc oxides, ferric ammonium ferrocyanide, manganese violet, ultramarine blue, and chrome oxide), organic powders, composite powders, optical brightener particles, and combinations thereof. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion.

### Other Skin Care Actives

The compositions of the present invention further may comprise non-vitamin antioxidants and radical scavengers, hair growth regulators, flavonoids, minerals, preservatives, phytosterols and/or plant hormones, protease inhibitors, tyrosinase inhibitors, anti-inflammatory agents and N-acyl amino acid compounds.

Suitable non-vitamin antioxidants and radical scavengers include, but are not limited to, BHT (butylated hydroxy toluene), L-ergothioneine (available as THIOTANE™); tetrahydrocurcumin, cetyl pyridinium chloride, camosine, diethylhexyl syrinylidene malonate (available as OXYNEX™), hexadec-8-ene-1,16-dicarboxylic acid (octadecene dioic acid; ARLATONE™ Dioic DCA from Uniqema), ubiquinone (co-enzyme Q10), tea extracts including green tea extract, yeast extracts or yeast culture fluid (e.g., Pitera®), and combinations thereof.

Suitable hair growth regulators include, but are not limited to, hexamidine, butylated hydroxytoluene (BHT), hexanediol, panthenol and pantothenic acid derivates, their isomers, salts and derivatives, and mixtures thereof.

Suitable minerals include zinc, manganese, magnesium, copper, iron, selenium and other mineral supplements. “Mineral” is understood to include minerals in various oxidation states, mineral complexes, salts, derivatives, and combinations thereof.

Suitable examples of plant sterols (phytosterols) and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, soybean trypsin inhibitor, Bowman-Birk inhibitor, and mixtures thereof.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, other licorice extracts, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include moisturizing and/or conditioning agents, such as glycerol, petrolatum, caffeine, and urea; yeast extracts (for example, Pitera™); dehydroepiandrosterone (DHEA), its analogs and derivatives; exfoliating agents, including alpha- and beta-hydroxyacids, alpha-keto acids, glycolic acid and octanoyl salicylate; antimicrobial agents; antidandruff agents such as piroctone olamine, 3,4,4′-trichlorocarbanilide(trichlosan), triclocarban and zinc pyrithione; dimethyl aminoethanol (DMAE); creatine; skin lightening agents such as kojic acid, mulberry extract, hydroquinone, arbutin, and deoxy-arbutin; (sunless) tanning agents, such as dihydroxy acetone (DHA); isomers, salts, and derivatives of any of the foregoing; and mixtures thereof.

### II. Methods of Use

The present invention describes a method of providing a benefit to mammalian keratinous tissue, including a method of protecting mammalian skin from the effects of ultraviolet radiation, comprising the step of topically applying to mammalian keratinous tissue a personal care composition described herein. Alternatively, the method may comprise the step of applying the composition described herein to insult-affected keratinous tissue, to regulate and/or improve the condition of such tissue, and/or to provide relief from the effects of the insult. Alternatively, the composition may be applied to skin exhibiting signs of skin aging, for example, to reduce the appearance of wrinkles.

Benefits include regulating and/or improving the condition of keratinous tissue, non-limiting examples of which include reducing the appearance of wrinkles, reducing the appearance of deep lines, reducing the appearance of fine lines, reducing the appearance of large pores, reducing the thickness of keratinous tissue, increasing the convolution of the dermal-epidermal border, increasing elasticity, reducing the appearance of cellulite, reducing the appearance of discoloration, reducing the appearance of hyperpigmentation, reducing the appearance of under-eye circles, reducing the appearance of sallowness, and combinations thereof. Alternatively, the benefit may include reducing wrinkles, reducing deep lines, reducing fine lines, reducing large pores, reducing cellulite, reducing hyperpigmentation, reducing undereye circles, reducing puffiness, and combinations thereof.

The composition may be applied by a variety of means, including by rubbing, wiping or dabbing with hands or fingers, or by means of an implement and/or delivery enhancement device. Non-limiting examples of implements include a sponge or sponge-tipped applicator, a swab (for example, a cotton-tipped swab), a pen optionally comprising a foam or sponge applicator, a lip balm, a brush, a wipe, and combinations thereof. Non-limiting examples of delivery enhancement devices include mechanical, electrical, ultrasonic and/or other energy devices. After application, the composition may be allowed to remain on the keratinous tissue.

The amount of the composition applied, the frequency of application and the period of use will vary widely depending upon the level of components of a given composition and the level of regulation desired. For example, from about 0.1 mg composition/cm² to about 50 mg composition/cm², and alternatively about 2 mg composition/cm² of keratinous tissue may be applied. In one embodiment, the composition is applied prior to exposure of the skin to ultraviolet radiation, and alternatively at least once daily, where “daily” and “days” mean a 24-hour period. The user may be instructed to reapply the composition after a period of time has passed, for example every hour, and alternatively when the composition has been washed or rinsed from the skin, for example after washing one's hands or after swimming. The composition further may be applied as part of a treatment regimen, for example, once daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

### III. Kit

The present invention further provides a kit comprising at least one composition described herein. The kit may comprise an outer packaging unit, which in turn may comprise one or more inner packaging units. In one embodiment, at least a portion of all packaging is transparent, such that the composition is visible to a consumer. One non-limiting example of a suitable outer container is a box or a tray, suitable for holding a sufficient number of inner packaging units for an indicated application regimen, for example, one application per day for one month. Alternatively, the tray may contain an array of individual inner packaging units which are organized to correspond to an indicated application regimen.

The inner packaging unit further may comprise indicators, for example, a stripe, arrow, or written instructions, to indicate to the consumer how much of the composition should be applied. For example, the consumer may be directed to dispense the amount of composition comprising one unit of the patterned anhydrous composition and apply to the skin prior to exposure to ultraviolet radiation and/or repeat after a given time of exposure has elapsed.

The kit further may comprise an implement, which may be suitable for targeted delivery of the composition to a desired area of keratinous tissue. The composition may be packaged separately from the implement, or may be contained within the implement. The kit further may comprise a plurality of components, including one or more additional compositions, one or more orally ingestible dietary supplements, an additional implement, an additional delivery enhancement device, a temperature change element, a substrate, instructions for complying with suitable application regimens, and combinations thereof.

### EXAMPLES 1-5

Aqueous gels, suitable for use as an aqueous phase in the composition of the present invention, may be made by conventional methods (by combining all ingredients and thoroughly mixing, as would be known to one of skill in the art) from the following components:

| | Exam- | Exam- | | Exam- | Exam- |
|---|---|---|---|---|---|
| | ple 1 | ple 2 | Example 3 | ple 4 | ple 5 |
| Water | q.s. | q.s. | q.s. | q.s. | q.s. |
| Glycerin | 12.5 | 10.0 | 20.0 | 3.0 | 10.0 |
| Pentylene Glycol | — | — | — | 5.0 | — |
| Niacinamide⁵ | 6.0 | — | 5.0 | 3.0 | 2.0 |
| N-Acetyl | — | 1.0 | 2.0 | — | — |
| Glucosamine⁵ | | | | | |
| Dex-panthenol | 0.5 | — | 1.0 | 0.3 | 0.5 |
| Sodium ascorbyl | — | — | — | 0.05 | — |
| Phosphate | | | | | |
| Hydroquinone | — | — | — | 2.0 | — |
| Carbopol | 0.7 | 0.85 | — | 1.2 | — |
| Ultrez ™-10¹ | | | | | |
| Carbopol | — | — | 0.7 | — | — |
| Ultrez ™-21² | | | | | |
| Sepigel ™ 305³ | — | — | — | — | 3.0 |
| Triethanolamine | 0.8 | 1.0 | 0.8 | 1.5 | — |
| Methylparaben | — | — | — | — | 0.05 |
| Glydant Plus Liquid⁴ | 0.3 | 0.3 | 0.3 | 0.2 | 0.1 |
| Disodium EDTA | 0.06 | 0.05 | 0.05 | 0.05 | 0.05 |
| Sodium Metabisulfite | — | — | — | 0.2 | — |
| FD&C Red 40 | — | — | 0.0002 | — | — |
| Total: | 100% | 100% | 100% | 100% | 100% |

¹Carbomer from Noveon ™
²Acrylates/C10–30 Alkyl Acrylate Crosspolymer from Noveon ™
³Polyacrylamide, C13–14 Isoparaffin, and Laueth-7 from Seppic ™
⁴DMDM Hydantoin and Iodopropynyl Butylcarbamate from Lonza ™
⁵Additionally or alternatively, the composition may comprise one or more other skin care actives, their salts and derivatives, as disclosed herein, in amounts also disclosed herein as would be deemed suitable by one of skill in the art.

### EXAMPLES 6-10

Anhydrous compositions, suitable for use as a non-aqueous phase in the composition of the present invention, may be prepared by conventional methods (by combining all ingredients and thoroughly mixing, as would be known to one of skill in the art) from the following components:

| | Exam- | Exam- | Exam- | | Exam- |
|---|---|---|---|---|---|
| | ple 6 | ple 7 | ple 8 | Example 9 | ple 10 |
| Octisalate | 20.0 | 20.0 | 10.0 | 20.0 | 20.0 |
| Homosalate | 20.0 | 20.0 | 25.0 | — | 10.0 |
| Octocrylene | 7.5 | 7.5 | 10.0 | 10.0 | 15.0 |
| Octinoxate | — | — | — | 25.0 | — |
| Avobenzone | 10.0 | 10.0 | 15.0 | — | 10.0 |
| Z-Cote HP1¹ | — | — | — | 10.0 | — |
| Isopropyl Isostearate | — | 10.0 | — | — | — |
| Isopropyl | 12.4 | — | 20.0 | — | 10.0 |
| Lauroylsarcosinate | | | | | |
| Phenylethyl benzoate | — | 10.0 | — | — | — |
| Vitamin E Acetate | — | 1.5 | — | 5.0 | — |
| Silica Dimethicone | 6.5 | 8.0 | 9.0 | — | — |
| Silylate | | | | | |
| Silica Silylate | — | — | — | 15.0 | 5.0 |
| Tospearl ™ 145A⁵ | 23.0 | 10.0 | — | 4.9 | — |
| Softouch ™ CC6098² | — | — | — | — | — |
| Fluoropure ™ 100C³ | — | 3.0 | 11.0 | 10.0 | 30.0 |
| Red Iron Oxide | — | — | — | 0.1 | — |
| Prestige ™ Fire Red⁴ | 0.1 | — | — | — | — |
| Perfume | 0.5 | — | — | — | — |
| Total: | 100% | 100% | 100% | 100% | 100% |

¹Zinc Oxide and Triethoxycaprylylsilane from BASF ™
²Boron Nitride powder from General Electric ™
³PTFE from Shamrock Technologies ™
⁴Mica and iron oxides from Eckart ™
⁵Polymethylsilsesquioxane from General Electric ™

### EXAMPLES 11-12

Oil-in-water skin care cream compositions, suitable for use as a third phase in the composition of the present invention, may be prepared by conventional methods (by combining all ingredients and thoroughly mixing, as would be known to one of skill in the art) from the following components:

| | Example 11 | Example 12 |
|---|---|---|
| Water Phase: | | |
| Water | q.s. | q.s. |
| Glycerin | 10.0 | 5.0 |
| Niacinamide³ | 5.0 | 2.0 |
| N-Acetyl Glucosamine | 2.0 | — |
| Pentylene Glycol | — | 1.0 |
| Triethanolamine | — | 0.37 |
| Dex-panthenol | 0.5 | 0.2 |
| Benzyl Alcohol | 0.25 | 0.25 |
| Sodium ascorbyl | 0.02 | — |
| Phosphate | | |
| Disodium EDTA | 0.05 | 0.05 |
| Methylparaben | 0.1 | 0.1 |
| Oil Phase | | |
| Sepiwhite ™ MSH¹ | — | 0.2 |
| Isohexadecane | 3.0 | 3.0 |
| Sucrose | 0.7 | — |
| Polycottonseedate | | |
| Cetyl Alcohol | 0.5 | 0.5 |
| Stearyl Alcohol | 0.5 | 0.5 |
| Behenyl Alcohol | 0.5 | 0.5 |
| Tospearl ™ 145A | 0.3 | — |
| Ethylparaben | 0.2 | 0.2 |
| PEG-100 Stearate | 0.1 | 0.1 |
| Propylparaben | 0.1 | 0.1 |
| Tocopheryl Acetate | 0.1 | 0.1 |
| Cetearyl Glucoside | 0.2 | 0.2 |
| Thickener | | |
| Sepigel ™ 305 | 2.5 | 2.5 |
| Carbomer Ultrez ™ 10 | — | 0.2 |
| Additional Ingredients | | |
| Dow Corning 1503 | 2.0 | — |
| Fluid² | | |
| Total: | 100% | 100% |

¹Undecylenoyl Phenylalanine from Seppic ™ (France)
²Dimethicone and Dimethiconol from Dow Corning ™
³Additionally or alternatively, the composition may comprise one or more other skin care actives, their salts and derivatives, as disclosed herein, in amounts also disclosed herein as would be deemed suitable by one of skill in the art.

In a suitable vessel, add the Carbomer Ultrez™ 10 to the water and mix until uniform. Next, add the rest of the water phase ingredients and heat to about 75° C. In a separate suitable vessel, combine the oil phase ingredients and heat to about 75° C. Add the oil phase to the water phase and mill the resulting emulsion (eg., with a Tekmar™ T-25 rotor-stator mil). Add the Sepigel™ thickener to the emulsion and cool the emulsion to about 45° C. while stirring. Add the remaining ingredients. Cool the product while stirring to about 30° C. and pour into suitable containers.

### EXAMPLES 13-14

Silicone elastomer compositions, suitable for use as a third phase in the composition of the present invention, may be prepared by conventional methods from the following components:

| | Example 13 | Example 14 |
|---|---|---|
| Cyclomethicone D5 | 19.8 | 18.8 |
| Dow Corning 9040¹ | — | 80 |
| Dow Corning 9045² | 70 | — |
| Tospearl 145A | 10 | — |
| Red Iron Oxide | 0.1 | — |
| Titanium Dioxide | — | 0.2 |
| Tocopheryl Acetate | 0.1 | 1.0 |
| Total: | 100% | 100% |

¹Cyclomethicone and Dimethicone Crosspolymer from Dow Corning ™
²Cyclomethicone and Dimethicone Crosspolymer from Dow Corning ™

The following represent some non-limiting examples of combinations of the above compositions to form a multi-phased composition:

### EXAMPLE 15

Combine any one of the compositions of Examples 1-5 with any one of the compositions of Examples 6-10, such that the ratio of the weight of the composition of Example 1-5 to the weight of the composition of Example 6-10 is about 9:1. Fill into transparent or translucent containers, such that the composition of Examples 6-10 forms a spiral shape within the composition from Examples 1-5.

### EXAMPLE 16

Combine any one of the compositions of Examples 1-5 with at least two of the compositions of Examples 6-10, such that the ratio of the weight of the composition of Examples 1-5 to the total weight of the compositions of Examples 6-10 is about 4:1. Fill into transparent or translucent containers, such that the compositions of Examples 6-10 form a multiple helix within the composition from Examples 1-5.

### EXAMPLE 17

Combine any one of the compositions of Examples 1-5 with at least one composition of Examples 6-10 and with any one of compositions of Examples 11-12, such that the ratio of the weight of the composition of Example 1-5 to the total weight of the compositions of Examples 6-10 and of Examples 11-12 is about 4:1. Fill into transparent or translucent containers, such that the compositions of Examples 6-10 and the compositions of Examples 11-12 form a swirled pattern together with the composition from Examples 1-5.

### EXAMPLE 18

Combine any one of the compositions of Examples 1-5 with at least one composition of Examples 6-10 and with any one of compositions of Examples 13-14, such that the ratio of the weight of the composition of Example 1-5 to the total weight of the compositions of Examples 6-10 and of Examples 13-14 is about 7:3. Fill into transparent or translucent containers, such that the compositions of Examples 6-10 and the compositions of Examples 11-12 form a marbled pattern together with the composition from Examples 1-5.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable personal care composition comprising a discrete transparent aqueous phase and a discrete non-aqueous phase, wherein:
a) the aqueous phase comprises from about 0.05% to about 5% of a polymeric thickener, and from about 50% to about99.5% water, both by weight of the aqueous phase;
b) the non-aqueous phase comprises from about 20% to about 90% of a sunscreen selected from the group consisting of oil-soluble sunscreens, oil-insoluble sunscreens, and combinations thereof; and from about 5% to about 40% of an oil thickener, all by weight of the non-aqueous phase; and
wherein the ratio of the weight of the aqueous phase to the weight of the non-aqueous phase is from about 1:1 to about 9:1; and wherein the aqueous phase and the non-aqueous phase are visually distinct and form a stable pattern.

2. The composition of claim 1, wherein the non-aqueous phase further comprises from about 0.001% to about 30% of a polar solvent.

3. The composition of claim 1, wherein at least one non-aqueous phase forms a stable pattern within the aqueous phase.

4. The composition of claim 3, wherein the average width of the non-aqueous phase is from about 1 mm to about 10 mm.

5. The composition of claim 3, wherein the pattern is selected from at least one continuous line, discontinuous line, curve, spiral, helix, and combinations thereof.

6. The composition of claim 1, wherein at least two non-aqueous phases form a stable pattern within the first phase.

7. The composition of claim 6, wherein the average width of each of the non-aqueous phases is from about 1 mm to about 10 mm.

8. The composition of claim 7, wherein the stable pattern is a multiple helix.

9. The composition of claim 1, wherein the aqueous phase and the non-aqueous phase together form a swirled pattern.

10. The composition of claim 1, wherein the aqueous phase and the non-aqueous phase together form a marbled pattern.

11. The composition of claim 1, wherein the aqueous phase further comprises from about 0.001% to about 5%, by weight of the aqueous phase, of a water-soluble sunscreen.

12. The composition of claim 1, wherein the aqueous phase further comprises at least one additional water-soluble skin care active.

13. The composition of claim 1, wherein the aqueous phase further comprises from about 0.001% to about 25%, by weight of the aqueous phase, of a conditioning agent.

14. The composition of claim 1, wherein the oil-soluble sunscreen is an organic sunscreen selected from the group consisting of benzophenone-3, bis-ethylhexyloxyphenol methoxyphenyl triazine, butyl methoxydibenzoyl-methane, diethylamino hydroxy-benzoyl hexyl benzoate, drometrizole trisiloxane, ethylhexyl methoxy-cinnamate, ethylhexyl salicylate, ethylhexyl triazone, octocrylene, homosalate, polysilicone-15, and derivatives and mixtures thereof.

15. The composition of claim 1, wherein the oil-insoluble sunscreen is selected from the group consisting of methylene bis-benzotriazolyl tetramethylbutyl-phenol, titanium dioxide, zinc cerium oxide, zinc oxide, and derivatives and mixtures thereof.

16. The composition of claim 1, wherein the oil thickener is selected from the group consisting of silica, hydrophobically-modified silica, fumed silica, hydrophobically-modified fumed silica, polytetrafluoroethylene, polyethylene, and derivatives and mixtures thereof.

17. The composition of claim 1, wherein the polar solvent is selected from the group consisting of di-isopropyl adipate, butyl and isopropyl phthalimide, phenylethyl benzoate, dicaprylyl carbonate, isopropyl lauroylsarcosinate, isononyl isononanoate, butyl octylsalicylate, dioctyl malate, dicaprylyl maleate, isopropyl isostearate, propylene glycol dicaprate, esters of C12-C15 alcohol benzoates, and derivatives and mixtures thereof.

18. The composition of claim 1, wherein the non-aqueous phase further comprises at least one additional skin care active.

19. The composition of claim 1, wherein the non-aqueous phase further comprises from about 0.001% to about 25% of colorant selected from the group consisting of organic pigments, inorganic pigments, natural dyes, synthetic dyes, lakes, hydrophilically-coated particulates, hydrophobically-coated particulates, interference pigments, and mixtures thereof.

20. The composition of claim 1, further comprising a discrete and visually distinct third phase.

21. The composition of claim 20, wherein the third phase comprises a silicone elastomer.

22. The composition of claim 21, wherein the third phase is anhydrous.

23. The composition of claim 20, wherein the third phase is an oil-in-water emulsion.

24. A method of providing a benefit to mammalian keratinous tissue, comprising the step of topically applying to mammalian keratinous tissue a stable personal care composition comprising a discrete transparent aqueous phase and a discrete non-aqueous phase, wherein:
a) the aqueous phase comprises from about 0.05% to about 5% of a polymeric thickener, and from about 50% to about 99.5% water, both by weight of the aqueous phase;
b) the non-aqueous phase comprises from about 20% to about 90% of a sunscreen selected from the group consisting of oil-soluble sunscreens, oil-insoluble sunscreens, and combinations thereof; and from about 5% to about 40% of an oil thickener, all by weight of the non-aqueous phase; and
wherein the ratio of the weight of the aqueous phase to the weight of the non-aqueous phase is from about 1:1 to about 9:1; and wherein the aqueous phase and the non-aqueous phase are visually distinct and form a stable pattern.

25. The method of claim 24, wherein the benefit is protecting the mammalian keratinous tissue from the effects of ultraviolet radiation.

26. The method of claim 24, wherein the mammalian keratinous tissue is insult-affected mammalian skin.

27. The method of claim 24, wherein the mammalian keratinous tissue is human skin exhibiting signs of skin aging.

28. A kit comprising:
a) a stable personal care composition comprising a discrete transparent aqueous phase and a discrete non-aqueous phase, wherein:
i. the aqueous phase comprises from about 0.05% to about 5% of a polymeric thickener, and from about 50% to about 99.5% water, both by weight of the aqueous phase;
ii. the non-aqueous phase comprises from about 20% to about 90% of a sunscreen selected from the group consisting of oil-soluble sunscreens, oil-insoluble sunscreens, and combinations thereof; and from about 5% to about 40% of an oil thickener, all by weight of the non-aqueous phase; and wherein the ratio of the weight of the aqueous phase to the weight of the non-aqueous phase is from about 1:1 to about 9:1; and wherein the aqueous phase and the non-aqueous phase are visually distinct and form a stable pattern; and,
b) at least one additional component select from the group consisting of at least one additional composition, at least one orally ingestible dietary supplement, an implement, a delivery enhancement device, a temperature change element, instructions for complying with suitable application regimens, and combinations thereof; and
c) instructions for complying with a regimen to provide a benefit to keratinous tissue.
